(19) United States
(12) Patent Application Publication
(10) Pub. No.: US 2019/0192573 A1
(43) Pub. Date: Jun. 27, 2019

(54) ANTI-OSTEOSARCOMA CAR-T DERIVED FROM THE ANTIBODY OI-3

(71) Applicant: Oncoinvent AS, Oslo (NO)

(72) Inventor: Tina Bjørnlund Bønsdorff, Oslo (NO)

(21) Appl. No.: 16/325,645

(22) PCT Filed: Aug. 18, 2017
(86) PCT No.: PCT/EP2017/070946

§ 371 Date: Feb. 14, 2019

(30) Foreign Application Priority Data
Aug. 19, 2016 (EP) 16184975.7

(51) Int. Cl. A61K 35/17; C12N 15/85; C07K 14/725; C12N 15/62; C07K 14/705; C07K 14/73; A61P 35/00; C07K 16/30; C12N 5/0783

(52) U.S. Cl. CPC A61K 35/17; C12N 15/85; C07K 14/7051; C12N 15/62; C12N 5/0646; C07K 14/70514; C07K 14/70517; A61P 35/00; C07K 16/3092; C07K 14/70521

(57) ABSTRACT

The invention relates to chimeric antigen receptor (CAR) specific to p80 and CD146, vectors encoding the same, and recombinant T cells comprising the p80 or CD146 CAR. The invention also includes methods of administering a genetically modified T cell expressing a CAR that comprises a p80 or CD146 binding domain.

### FIELD OF THE INVENTION

The present invention relates to chimeric antigen receptor (CAR) specific to p80 or CD146, vectors encoding the same, and recombinant T cells comprising the p80 or CD146 CAR. The invention also includes methods of administering a genetically modified T cell expressing a CAR that comprises a p80 or CD146 binding domain.

### BACKGROUND OF THE INVENTION

The use of chimeric antigen receptor (CAR) T-cell (CAR-T) immunotherapy against cancer in general and also against osteosarcoma has been widely studied (DeRenzo et al., Adv Exp Med Biol. 2014; 804: 323-340).

The antibodies TP-1 and TP-3 have been described previously (Olafsen et al., Acta Oncol. 1996; 35(3):297-301, Olafsen et al., Nucl Med Biol. 1995 August; 22(6):765-71, Olafsen et al., Cancer Immunol Immunother. 1999 October; 48(7):411-8, Onda et al., J Immunother (1991). 2001 March; 24(2):144-150, and Onda et al., J Immunother. 2001 March-April; 24(2):144-50) and the target alkaline phosphatase isoenzyme seems to have promising expression level (Bruland et al., Cancer Res. 1988 Sep. 15; 48(18):5302-9.).

The OI-3 antibody is well-known and described in PCT/EP2014/070395.

There is a great need for new and better approaches to fight cancer, including osteosarcoma.

### SUMMARY OF THE INVENTION

An aspect of the present invention relates to a nucleic acid molecule encoding a polypeptide comprising one or more sequences selected from the group consisting of a) SEQ ID NO.: 1, b) SEQ ID NO.: 2, c) SEQ ID NO.: 3, d) SEQ ID NO.: 4, e) SEQ ID NO.: 5, f) SEQ ID NO.: 6, g) a polypeptide capable of specifically binding p80 or CD146, h) a sequence with at least 80% sequence identity with the sequence of a), b), c), d), e), f) or g), and i) a fragment of any one of a)-h).

The nucleic acid molecule may be RNA or DNA.

An embodiment of the present invention relates to the nucleic acid molecule according to the invention which is a CAR-T construct.

A further embodiment of the present invention relates to the nucleic acid molecule according to the invention, which is a CAR-T construct selected from the group consisting of a transient mRNA construct and a transduction vector.

A further aspect of the present invention relates to a host cell comprising the nucleic acid molecules of the present invention.

An embodiment of the present invention relates to the host cell according to present invention, which is a T-cell.

Another embodiment of the present invention relates to host cell according to the present invention, which is a recombinant T-cell.

A further embodiment of the present invention relates to host cell of the present invention, which is expressing a chimeric antigen receptor (CAR) specific to p80 or CD146.

Another aspect of the present invention relates to a method for treating osteosarcoma, comprising administering a therapeutically effective number of cells according to the present invention to an individual in need thereof.

Another aspect of the present invention relates to a nucleic acid molecule according to the present invention for use as a medicament.

A further aspect of the present invention relates to the nucleic acid molecule according to the present invention for use in the treatment of cancer.

Another aspect of the present invention relates to a host cell according to the present invention for use as a medicament.

Another embodiment of the present invention relates to the host cell according to the present invention for use in the treatment of cancer.

In an embodiment of the present invention is the cancer osteosarcoma.

In an embodiment of the present invention is administration done parenterally.

An aspect of the present invention relates to a chimeric antigen receptor (CAR) linked to antibody or antibody-fragment.

In an embodiment of the present invention is CAR specific to p80 or CD146.

In an embodiment of the present invention is the antibody or antibody-fragment selected from the group consisting of TP-1 and TP-3, any other p80 binding antibody or antibody-fragment, OI-3 or any other CD146 binding antibody or antibody-fragment.

### DETAILED DESCRIPTION OF THE INVENTION

The inventor has surprisingly found that the antigens recognized by TP-1 and TP-3 (p80 on osteosarcoma cells) and OI-3 (anti-CD146) are ideal targets for CAR-T immunotherapy.

The p80 antigen is suitable for in vitro and in vivo targeting with immunotoxins and radioimmunoconjugates as well as complement mediated therapy and is also suitable for use in CAR-T mediated immunotherapy (U.S. Pat. No. 6,042,829, Larsen et al., Br J Cancer. 1994 June; 69(6):1000-5, Ek et al., Clin Cancer Res. 1998 July; 4(7):1641-7).

The variable sequences of TP-1 and TP-3 have been sequenced and published several places, including Olafsen et al., Actan Oncologica vol. 35, no. 3, 297-301 and Olafsen et al., Nucl. Med. Biol. Vol. 22, no. 6, 765-771.

The sequences are:

TP-1 variable light chain sequence (SEQ ID NO.:
1):
DIELTQSPAIMSASPGEKVTMTCSASSSVSYMHWYQQKSGTSPKRWIYDT
SKLASGVPARFSGSGSGTSYSLTISSVEAEDAATYYCQQYSGHPLTFGAG
TKLELKR
TP-1 variable light chain is described in GenBank:
AJ131747.1
TP-1 variable heavy chain sequence (SEQ ID NO.:
2):
EVQLQESGPSLVKPSQTLSLTCSVTGDSITSGYWNWIRKFPGNKLEYMGY
ISYSDTTYYNPSLKSRISITRDTSKNQYYLHLKSVTTEDTATYYCASAYY
GSSLSMGNWGQGTSATVSS
TP-1 variable heavy chain is described in GenBank:
AJ131748.1.
TP-3 variable light chain sequence (SEQ ID NO.:
3):
DIELTQSPASLAVSLGQRATISCRASKSVSTGYSYLHWYQQKPGQPPKLL
IYLASNLESGVPARFSGSGSGTDFTLNIHPVEEEDAATYYCQHSRELPLT
FGAGTKLEIKR
TP-3 variable light chain is described in GenBank:
AJ131749.1
TP-3 variable heavy chain sequence (SEQ ID NO.:
4):
RIQLQQSGAELVKPGASVKISCKASGYIFTDYNMDWVKQSHGKSLEWIGD
INPNYDSTRYNQKFKGKATLTVDKSSSTAYMELRSLTSEDTAVYYCARGD
YYVSSYGHDYAMDYWGQGTTVTVSS
TP-3 variable heavy chain is described in GenBank:
AJ131750.1
OI-3 variable light chain sequence (SEQ ID NO.:
5):
MKLPVRLLVLMFWIPASSSDVVMTQTPLSLPVSLGDQASISCRSSQSLVH
SNGNTYLHWYLQKPGQSPKLLIYKVSNRFSGVPDRFSGSGSGTDFTLKIS
RVEAEDLGVYFCSQSTHVSTFGGGTKLEIK
OI-3 variable heavy chain sequence (SEQ ID NO.:
6):
MGWIWFFLFLLSGTAGVHSEVQLQQSGPELVKTGASVKISCKASGYSFTG
YYIHWVKQSHGKSLEWIGYISNYNGATTYSQEFKGKATFTVDRSSRIAYM
QFTGLTSEDSAVYYCAGNSWGDWYFDVWGAGTTVTVSS

The OI-3 antibody is well-known and the sequences are disclosed in PCT/EP2014/070395.

An aspect of the present invention relates to a nucleic acid molecule encoding a polypeptide comprising one or more sequences selected from the group consisting of a) SEQ ID NO.: 1, b) SEQ ID NO.: 2, c) SEQ ID NO.: 3, d) SEQ ID NO.: 4, e) SEQ ID NO.: 5, f) SEQ ID NO.: 6, g) a polypeptide capable of specifically binding p80 or CD146, h) a sequence with at least 80% sequence identity with the sequence of a), b), c), d), e), f) or g), and i) a fragment of any one of a)-h).

The nucleic acid molecule may be DNA or RNA.

Another aspect of the present invention relates to a polypeptide sequence comprising one or more sequences selected from the group consisting of a) SEQ ID NO.: 1, b) SEQ ID NO.: 2, c) SEQ ID NO.: 3, d) SEQ ID NO.: 4, e) SEQ ID NO.: 5, f) SEQ ID NO.: 6, g) a polypeptide capable of specifically binding p80 or CD146, h) a sequence with at least 80% sequence identity with the sequence of a), b), c), d), e), f) or g), and i) a fragment of any one of a)-h).

An aspect of the present invention relates to a nucleic acid molecule encoding a chimeric antigen receptor (CAR) directed against the antigen CD146 or p80, wherein said CAR when expressed on the surface of an immune effector cell is capable of binding to the antigen CD146 or p80 expressed on a target cell surface and comprises an antigen-binding domain comprising a VL sequence and a VH sequence defined by: a) SEQ ID NOs. 5 and 6 for CD146; or b) SEQ ID NOs. 3 and 4 for p80, or c) SEQ ID NOs. 1 and 2 for p80.

One embodiment of the present invention relates to nucleic acid molecule of the present invention, wherein the antigen-binding domain is a scFv comprising the VL and VH sequences.

The antigen-binding domain or scFV may comprise, in the following order, the VL sequence, a linker sequence, and the VH sequence. The linker sequence may be (G4S)4. The CAR can comprise a plasma membrane targeting sequence. The plasma membrane targeting sequence can be positioned upstream of the antigen-binding domain or scFv.

In one embodiment of the present invention the CAR comprises, downstream of an extracellular domain comprising the antigen-binding domain, a hinge domain, a transmembrane domain, an intracellular signaling domain, and optionally one or more co-stimulatory signaling domains.

The hinge domain may be derived from CD8a, CD4, CD28, or CD7, preferably human CD8a, CD4, CD28, or CD7, or from the Fc of an immunoglobulin, for example IgG, preferably wherein the Fc-derived hinge domain does not comprise a CH3 domain, e.g. comprises or consists of the CH2 domain or a part thereof.

The transmembrane domain of the CAR may be a transmembrane domain derived from CD8a, 003, CD28, CD4, CD45, CD9, CD16, CD22, CD33, CD64, CD80, CD86, CD134, CD137, or CD154, preferably human CD8a, CD3, CD28, or CD4.

An embodiment of the present invention relates to a vector comprising the nucleic acid molecule of the present invention.

As aspect relates to a cell comprising the nucleic acid molecules and/or the vectors of the present invention. The cell may be an immune effector cell. Thus, an aspect of the present invention relates to an immune effector cell comprising the nucleic acid molecule and/or the vector of the present invention.

The cell may be a T-cell or an NK cell.

An aspect of the present invention relates to a composition comprising the immune effector cell of the present invention and at least one physiologically acceptable carrier or excipient.

The immune effector cell and/or a composition of the present invention may be used in therapy or as a medicament. As commonly defined “identity” is here defined as sequence identity between genes or proteins at the nucleotide or amino acid level, respectively.

Thus, in the present context “sequence identity” is a measure of identity between proteins at the amino acid level and a measure of identity between nucleic acids at nucleotide level.

The protein sequence identity may be determined by comparing the amino acid sequence in a given position in each sequence when the sequences are aligned.

Similarly, the nucleic acid sequence identity may be determined by comparing the nucleotide sequence in a given position in each sequence when the sequences are aligned.

To determine the percent identity of two nucleic acid sequences or of two amino acids, the sequences are aligned for optimal comparison purposes (e.g., gaps may be introduced in the sequence of a first amino acid or nucleic acid sequence for optimal alignment with a second amino or nucleic acid sequence). The amino acid residues or nucleotides at corresponding amino acid positions or nucleotide positions are then compared.

When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=# of identical positions/total # of positions (e.g., overlapping positions)×100). In one embodiment the two sequences are the same length.

One may manually align the sequences and count the number of identical nucleic acids or amino acids. Alternatively, alignment of two sequences for the determination of percent identity may be accomplished using a mathematical algorithm. Such an algorithm is incorporated into the NBLAST and XBLAST programs. BLAST nucleotide searches may be performed with the NBLAST program, score=100, wordlength=12, to obtain nucleotide sequences homologous to a nucleic acid molecules of the invention. BLAST protein searches may be performed with the XBLAST program, score=50, wordlength=3 to obtain amino acid sequences homologous to a protein molecule of the invention.

To obtain gapped alignments for comparison purposes, Gapped BLAST may be utilised. Alternatively, PSI-Blast may be used to perform an iterated search which detects distant relationships between molecules. When utilising the NBLAST, XBLAST, and Gapped BLAST programs, the default parameters of the respective programs may be used. See http://www.ncbi.nlm.nih.gov. Alternatively, sequence identity may be calculated after the sequences have been aligned e.g. by the BLAST program in the EMBL database (www.ncbi.nlm.gov/cgi-bin/BLAST).

Generally, the default settings with respect to e.g. “scoring matrix” and “gap penalty” may be used for alignment. In the context of the present invention, the BLASTN and PSI BLAST default settings may be advantageous.

The percent identity between two sequences may be determined using techniques similar to those described above, with or without allowing gaps. In calculating percent identity, only exact matches are counted.

In an embodiment of the present invention relates to polynucleotides (nucleic acid molecules) encoding or polypeptides that have a sequence identity with the sequences according to a) SEQ ID NO.: 1, b) SEQ ID NO.: 2, c) SEQ ID NO.: 3, d) SEQ ID NO.: 4 e) SEQ ID NO.: 5 f) SEQ ID NO.: 6 of 80%, such as 85%, such as 90%, such as 95%, such as 98%, such as 99% and capable of binding to p80 or CD146.

In an embodiment is the sequence identity less than 100% indicating that at least one amino acid has been substituted, deleted, or inserted.

An embodiment of the present invention relates to the nucleic acid molecule according to the invention which is a CAR-T construct (CAR).

A further embodiment of the present invention relates to the nucleic acid molecule according to the invention, which is a CAR-T construct selected from the group consisting of a transient mRNA construct and a transduction vector.

First generation CARs typically had the intracellular domain from the CD3ζ-chain, which is the primary transmitter of signals from endogenous TCRs. Second generation CARs add intracellular signaling domains from various costimulatory protein receptors (e.g., CD28, 41BB, ICOS) to the cytoplasmic tail of the CAR to provide additional signals to the T cell. Preclinical studies indicated that the second generation improves the antitumor activity of T cells. More recent, third generation CARs combine multiple signaling domains, such as CD3z-CD28-41BB or CD3z-CD28-OX40, to augment potency.

The evolution of CAR therapy is an excellent example of the application of basic research to the clinic. The PI3K binding site used was identified in co-receptor CD28, while the

ITAM motifs were identified as a target of the CD4- and CD8-p56lck complexes.

Thus, in one embodiment of the CAR design a being a first generation CAR.

In a further embodiment is the CAR design a second CAR.

In a further embodiment is the CAR design a third generation CAR.

The introduction of Strep-tag II sequence (an eight-residue minimal peptide sequence (Trp-Ser-His-Pro-Gln-Phe-Glu-Lys) that exhibits intrinsic affinity toward streptavidin) into specific sites in synthetic CARs or natural T-cell receptors provides engineered T cells with an identification marker for rapid purification, a method for tailoring spacer length of chimeric receptors for optimal function and a functional element for selective antibody-coated, microbead-driven, large-scale expansion. Strep-tag can be used to stimulate the engineered cells, causing them to grow rapidly. Using an antibody that binds the Strep-tag, the engineered cells can be expand by 200-fold. Unlike existing methods this technology stimulates only cancer-specific T cells.

Thus, in an embodiment of the present invention is a Step-tag introduced. In another embodiment is a FLAG-tag introduced (see example 3).

SMDCs (small molecule drug conjugates) platform in immuno-oncology is a novel (currently experimental) approach that makes possible the engineering of a single universal CAR T cell, which binds with extraordinarily high affinity to a benign molecule designated as FITC. These cells are then used to treat various cancer types when co-administered with bispecific SMDC adaptor molecules. These unique bispecific adaptors are constructed with a FITC molecule and a tumor-homing molecule to precisely bridge the universal CAR T cell with the cancer cells, which causes localized T cell activation. Anti-tumor activity in mice is induced only when both the universal CAR T cells plus the correct antigen-specific adaptor molecules are present. Anti-tumor activity and toxicity can be controlled by adjusting the administered adaptor molecule dosing. Treatment of antigenically heterogeneous tumors can be achieved by administration of a mixture of the desired antigen-specific adaptors. Thus, several challenges of current CAR T cell therapies, such as: the inability to control the rate of cytokine release and tumor lysis, the absence of an “off switch” that can terminate cytotoxic activity when tumor eradication is complete, a requirement to generate a different CART cell for each unique tumor antigen, may be solved or mitigated using this approach.

An aspect of the present invention relates to a chimeric antigen receptor (CAR) linked to antibody or antibody-fragment.

In an embodiment of the present invention is CAR specific to p80 antigen on osteosarcoma cells.

In an embodiment of the present invention is the molecule of the present invention a polypeptide that is designed as a SMDC.

In an embodiment of the present invention is the antibody or antibody-fragment selected from the group consisting of TP-1 and TP-3, any other p80 binding antibody or antibody-fragment, OI-3 or any other CD146 binding antibody or antibody-fragment.

Also versions using transiently redirected t-cells of CAR T as described are included in the present invention. These includes Almåsbak et al., Gene Ther. 2015 May; 22(5):391-403. doi: 10.1038/gt.2015.4. Epub 2015 Feb 5 and Mensali et al., Oncoimmunology. 2016 Feb. 18; 5(5):e1138199. doi: 10.1080/2162402X.2016.1138199. eCollection 2016, and WO2017118745.

### Cells

A further aspect of the present invention relates to a host cell comprising the nucleic acid molecules of the present invention.

An embodiment of the present invention relates to the host cell according to present invention, which is a T-cell.

Another embodiment of the present invention relates to host cell according to the present invention, which is a recombinant T-cell.

A further embodiment of the present invention relates to host cell of the present invention, which is expressing a chimeric antigen receptor (CAR) specific to p80 or CD146.

### Methods and Uses

Another aspect of the present invention relates to a method for treating osteosarcoma, comprising administering a therapeutically effective number of cells according to the present invention to an individual in need thereof.

Another aspect of the present invention relates to a nucleic acid molecule according to the present invention for use as a medicament.

A further aspect of the present invention relates to the nucleic acid molecule according to the present invention for use in the treatment of cancer.

Another aspect of the present invention relates to a host cell according to the present invention for use as a medicament.

Another embodiment of the present invention relates to the host cell according to the present invention for use in the treatment of cancer.

The cancer types that can be treated are selected from the types where p80 and/CD146 is expressed.

In an embodiment of the present invention is the cancer osteosarcoma.

In another embodiment is the cancer selected from the group consisting of melanoma, pancreas, tipple negative breast cancer, and other cancer types where CD146 is expressed.

In an embodiment of the present invention is administration done parenterally.

Combinational treatment with other antigens is also an aspect of the present invention. This combination can a combination of different CARs targeting different antigens, or a mix of one or more CARs and antibodies or antibodies linked to CAR-Ts or SMDCs.

### EXAMPLES

### Example 1—Construction of Transient CAR Using TP-1, TP-3 and OI-3 Variable Sequences

Transient CAR is done in several different approaches. One approach is described in PCT/EP2015/062933 and also in Inderberg et al. (poster, abstract 2310, AACR).

Other approaches are described in Almåsbak et al., Gene Ther. 2015 May; 22(5):391-403. doi: 10.1038/gt.2015.4. Epub 2015 Feb 5 and Mensali et al., Oncoimmunology. 2016 Feb 18;5(5):e1138199. doi: 10.1080/2162402X.2016.1138199. eCollection 2016.

The variable sequences of TP-1, TP-3 and OI-3 are used in the generation of mRNA based CARs.

It is surprising to see the effect the CARs based on TP-1, TP-3 and OI-3 have on osteosarcoma cells. This shows that CARs based on TP-1 and TP-3 can kill osteosarcoma cells and are candidates for treatment of cancer such as osteosarcoma.

### Example 2—Construction of Transduced CAR Using TP-1, TP-3 and OI-3 Variable Sequences

Transduced CAR is done in different ways a described above (first, second or third generation). Examples includes Kochenderfer et al., Blood, 11 Nov. 2010, Vol. 116, no 19, DeRenzo et al., Adv Exp Med Biol. 2014; 804: 323-340.

The variable sequences of TP-1, TP-3 and OI-3 are used in the generation of transduction based CARs.

It is surprising to see the effect the CARs based on TP-1, TP-3 and OI-3 has on osteosarcoma cells. This shows that CARs based on TP-1, TP-3 and OI-3 can kill osteosarcoma cells and are candidates for treatment of cancer such as osteosarcoma.

### Example 3—Construction of Transduced CAR Against p80 or CD146 using TP-1, TP-3 and OI-3 Sequences

CAR Constructs The mouse anti-p80 and anti CD146 scFv (a fusion protein of the variable regions of the heavy (VH) and light chains (VL) according to SEQ ID Nos.: 1-2, 3-4, and 3-4, connected with a short linker peptide of ten to about 25 amino acids) is inserted into a second-generation CAR cassette containing a signaling peptide from GM-CSF, a hinge region, transmembrane domain and costimulatorydomain from CD28, and the CD3ζ activation domain; these CARs are the p80 and the CD146 CAR.

The FLAG tag (DYKDDDDK) is inserted into the CARs between the scFv and hinge region; these CARs are called the the p80-FLAG CAR and the CD146-FLAG CAR.

### Generation of CAR-Encoding Lentivirus

DNAs encoding the CARs are synthesized and subcloned into a third-generation lentiviral vector, Lenti CMV-MCS-EF1a-puro by Syno Biological (Beijing, China). All CAR lentiviral constructs are sequenced in both directions to confirm CAR sequence and used for lentivirus production. Ten million growth-arrested HEK293FT cells (Thermo Fisher) are seeded into T75 flasks and cultured overnight, then transfected with the pPACKH1 Lentivector Packaging mix (System Biosciences, Palo Alto, Calif.) and 10 μg of each lentiviral vector using the CalPhos Transfection Kit (Takara, Mountain View, Calif.). The next day the medium was replaced with fresh medium, and 48 h later the lentivirus-containing medium are collected.

The medium was cleared of cell debris by centrifugation at 2100 g for 30 min. The virus particles are collected by centrifugation at 112,000 g for 100 min, suspended in AIM V-AlbuMAX medium (Thermo Fisher), aliquoted and frozen at −80° C. The titers of the virus preparations are determined by quantitative RT-PCR using the Lenti-X qRT-PCR kit (Takara) and the 7900HT thermal cycler (Thermo Fisher). The lentiviral titers are >1×108 pfu/ml. Lentiviruses are generated and used in accordance with approved biosafety level-2 regulations.

### Generation and Expansion of CAR-T Cells

PBMC are isolated from human peripheral blood buffy coats (provided by the Stanford University Blood Center in accordance with its approved IRB protocol) suspended at 1×106 cells/ml in AIM V medium containing 10% FBS and 300 U/ml IL-2 (Thermo Fisher), mixed with an equal number (1:1 ratio) of CD3/CD28 Dynabeads (Thermo Fisher), and cultured in non-treated 24-well plates (0.5 ml per well).

At 24 and 48 hours, lentivirus are added to the cultures at a multiplicity of infection

(MOI) of 5, along with 1 ml of TransPlus transduction enhancer (AlStem). As the T cells proliferated over the next two weeks, the cells are counted every 2-3 days and fresh medium with 300 U/ml IL-2 are added to the cultures to maintain the cell density at 1-3×10⁶ cells/ml.

An exact protocol can be found in: Frontiers In Bioscience, Landmark, 22, 1644-1654, Jun. 1, 2017.

### Example 4—The Design and Construction and Testing of Anti-146 and Anti-p80 CARs

### MATERIALS AND METHODS

CD146 and p80 CARs are designed to contain an antigen-binding part (scFv) according to the variable sequences according to SEQ ID Nos.: 1-2, 3-4 and 5-6.

The exact sequences and procedures are disclosed in WO2017118745, which hereby is incorporated herein by reference.

1. A nucleic acid molecule comprising a sequence encoding a chimeric antigen receptor (CAR) directed against the antigen CD146, wherein said CAR when expressed on the surface of an immune effector cell is capable of binding to the antigen CD146 expressed on a target cell surface and, wherein said CAR comprises an antigen-binding domain comprising a VL sequence and a VH sequence defined by:
a) SEQ ID NOs. 5 and 6.

2-20. (canceled)

21. The nucleic acid molecule of claim 1, wherein the antigen-binding domain is a scFv comprising the VL and VH sequences.

22. The nucleic acid molecule of claim 1, wherein the antigen-binding domain or scFV comprises, in the following order, the VL sequence, a linker sequence, and the VH sequence.

23. The nucleic acid molecule of claim 22, wherein the linker sequence is (G4S)4.

24. The nucleic acid molecule of claim 1, wherein the CAR comprises a plasma membrane targeting sequence.

25. The nucleic acid molecule of claim 24, wherein said plasma membrane targeting sequence is positioned upstream of the antigen-binding domain or scFv.

26. The nucleic acid molecule of claim 1, wherein the CAR comprises a hinge domain, a transmembrane domain, an intracellular signaling domain and optionally, one or more co-stimulatory signaling domains, downstream of an extracellular domain comprising the antigen-binding domain.

27. The nucleic acid molecule of claim 26, wherein the hinge domain is derived from CD8a, CD4, CD28, or CD7 or from a Fc of an immunoglobulin, wherein said Fc-derived hinge domain does not comprise a CH3 domain.

28. The nucleic acid molecule of claim 26, wherein the transmembrane domain of the CAR is a transmembrane domain derived from CD8a, CD3ζ, CD28, CD4, CD45, CD9, CD16, CD22, CD33, CD64, CD80, CD86, CD134, CD137, or CD154.

29. The nucleic acid molecule of claim 1, wherein said nucleic acid molecule is RNA.

30. A vector comprising the nucleic acid molecule of claim 1.

31. An immune effector cell comprising the nucleic acid molecule of claim 1.

32. The immune effector cell of claim 31, wherein the cell is a T-cell or an NK cell.

33. A composition comprising the immune effector cell of claim 31 and at least one physiologically acceptable carrier or excipient.

34. A method of adoptive cell transfer comprising administering the immune effector cell of claim 31 to a subject in need thereof.

35. A method for inhibiting a cancer comprising administering the immune effector cell of claim 31 to a subject in need thereof.

36. The method as claimed in claim 36, wherein the cancer is a B-cell malignancy.

37. A nucleic acid molecule comprising one or more sequences encoding a polypeptide selected from the group consisting of:
e) SEQ ID NO.: 5,
f) SEQ ID NO.: 6,
g) a polypeptide capable of specifically binding CD146,
h) a sequence with at least 80% sequence identity with the sequence of e), f) or g), and
i) a fragment of any one of e)-h).

38. The nucleic acid molecule according to claim 37, which is a CAR-T construct.


SEQUENCE LISTING (6 sequences)

SEQ ID NO: 1 - protein, 107 aa, Mus musculus
DIELTQSPAIMSASPGEKVTMTCSASSSVSYMHWYQQKSGTSPKRWIYDTSKLASGVPARFSGSGSGTSYSLTISSVEAEDAATYYCQQYSGHPLTFGAGTKLELKR

SEQ ID NO: 2 - protein, 119 aa, Mus musculus
EVQLQESGPSLVKPSQTLSLTCSVTGDSITSGYWNWIRKFPGNKLEYMGYISYSDTTYYNPSLKSRISITRDTSKNQYYLHLKSVTTEDTATYYCASAYYGSSLSMGNWGQGTSATVSS

SEQ ID NO: 3 - protein, 111 aa, Mus musculus
DIELTQSPASLAVSLGQRATISCRASKSVSTGYSYLHWYQQKPGQPPKLLIYLASNLESGVPARFSGSGSGTDFTLNIHPVEEEDAATYYCQHSRELPLTFGAGTKLEIKR

SEQ ID NO: 4 - protein, 125 aa, Mus musculus
RIQLQQSGAELVKPGASVKISCKASGYIFTDYNMDWVKQSHGKSLEWIGDINPNYDSTRYNQKFKGKATLTVDKSSSTAYMELRSLTSEDTAVYYCARGDYYVSSYGHDYAMDYWGQGTTVTVSS

SEQ ID NO: 5 - protein, 130 aa, Mus musculus
MKLPVRLLVLMFWIPASSSDVVMTQTPLSLPVSLGDQASISCRSSQSLVHSNGNTYLHWYLQKPGQSPKLLIYKVSNRFSGVPDRFSGSGSGTDFTLKISRVEAEDLGVYFCSQSTHVSTFGGGTKLEIK

SEQ ID NO: 6 - protein, 138 aa, Mus musculus
MGWIWFFLFLLSGTAGVHSEVQLQQSGPELVKTGASVKISCKASGYSFTGYYIHWVKQSHGKSLEWIGYISNYNGATTYSQEFKGKATFTVDRSSRIAYMQFTGLTSEDSAVYYCAGNSWGDWYFDVWGAGTTVTVSS